(19) United States
(12) Patent Application Publication
(10) Pub. No.: US 2008/0132743 A1
(43) Pub. Date: Jun. 5, 2008

(54) PRODUCTION OF HIGH PURITY DECABROMODIPHENYLALKANES

(75) Inventors: Arthur G. Mack, Prairieville, LA (US); Saadat Hussain, Baton Rouge, LA (US)

Correspondence Address: McGLINCHEY STAFFORD, PLLC, 4703 BLUEBONNET BLVD, BATON ROUGE, LA, 70809, US

(73) Assignee: ALBEMARLE CORPORATION, Baton Rouge, LA (US)

(21) Appl. No.: 11/840,318

(22) Filed: Aug. 17, 2007

Related U.S. Application Data
(60) Provisional application No. 60/868,242, filed on Dec. 1, 2006.

(51) Int. Cl. C07C 17/10

(52) U.S. Cl. 570/206

(57) ABSTRACT

Reaction-derived decabromodiphenylalkane product, especially decabromodiphenylethane product, of high purity is formed by (A) maintaining in a loop reactor a circulating inventory comprising at least Lewis acid bromination catalyst and excess liquid bromine; (B) introducing diphenylalkane and/or partially brominated diphenylalkane into the reactor so that bromination can occur; and (C) after a period of travel in the reactor during which solids of reaction-derived decabromodiphenylalkane product of high purity is formed, removing such solids from the reactor.

### REFERENCE TO RELATED APPLICATION

This application claims the benefit and priority of U.S. Provisional Application No. 60/868,242, filed Dec. 1, 2006, the disclosure of which is incorporated herein by reference.

### TECHNICAL FIELD

This invention relates to the preparation of high purity decabromodiphenylalkane products such as decabromodiphenylethane products.

### BACKGROUND

Decabromodiphenylethane (DBDPE) is a time-proven flame retardant for use in many flammable macromolecular materials, e.g. thermoplastics, thermosets, cellulosic materials and back coating applications.

DBDPE is presently sold as a powder derived from the bromination of 1,2-diphenylethane (DPE). Among prior processes for effecting such bromination are the bromination processes described in U.S. Pat. Nos. 6,518,468; 6,958,423; 6,603,049; 6,768,033; and 6,974,887. While it has been possible in the past to produce very high purity DBDPE, this has not been accomplished on a consistent basis. Accordingly, it would be desirable if process technology could be provided that would enable the production of highly pure DBDPE or its homologs on a consistent basis.

### BRIEF SUMMARY OF THE INVENTION

This invention is deemed to enable production of high purity decabromodiphenylalkane products without recourse to recrystallization or chromatographic purification steps or any other subsequent procedure to remove or that removes nonabromodiphenylalkane from decabromodiphenylalkane such as decabromodiphenylethane. In addition, this invention is deemed to enable production of highly pure DBDPE on a consistent basis.

Among the embodiments of this invention is a process for producing a reaction-derived decabromodiphenylalkane product of high purity, which process comprises:
A) maintaining in a loop reactor a circulating inventory comprising at least liquid bromine and Lewis acid bromination catalyst;
B) introducing diphenylalkane and/or partially brominated diphenylalkane into said loop reactor at a reactor inlet portion so that bromination occurs in said loop reactor; and
C) after a period of travel in said loop reactor that enables formation therein of reaction-derived decabromodiphenylalkane product of high purity in the form of solids, removing from an outlet portion in the loop reactor a portion of the circulating inventory containing at least some of said solids, and having the remaining portion of the circulating inventory continue flowing in the loop reactor.

A preferred embodiment of this invention is a process for preparing reaction-derived decabromodiphenylethane of high purity, which process comprises:
A) introducing diphenylethane or partially brominated diphenylethane, or both, into a loop reactor containing a circulating inventory comprising at least (a) liquid bromine, (b) Lewis acid bromination catalyst and optionally (c) bromination reaction products formed by bromination of said partially brominated diphenylethane;
B) separating a portion of the circulating inventory from said reactor at a remote locus downstream from the locus of introduction of the diphenylethane or partially brominated diphenylethane, or both, and recovering reaction-derived product solids from the separated portion of the circulating inventory and returning to the loop reactor, liquid from which said solids have been removed, and
C) replenishing bromine and Lewis acid bromination catalyst in the circulating inventory so as to maintain therein (i) an excess of bromine relative to the incoming diphenylethane and (ii) a catalytic quantity of Lewis acid bromination catalyst.

Another preferred embodiment of this invention is a process for preparing reaction-derived decabromodiphenylethane of high purity, which process comprises:
A) introducing diphenylethane or partially brominated diphenylethane, or both, into a loop reactor containing a circulating inventory comprising at least liquid bromine and Lewis acid bromination catalyst;
B) recovering decabromodiphenylethane product solids from the circulating inventory at a remote locus downstream from the locus of introduction of diphenylethane or partially brominated diphenylethane, or both, and enabling liquid from which said solids have been removed to continue flowing in the reactor as part of the circulating inventory contained within the reactor; and
C) replenishing bromine and Lewis acid bromination catalyst in the circulating inventory so as to maintain therein (i) an excess of bromine relative to the incoming diphenylethane and (ii) a catalytic quantity of Lewis acid bromination catalyst.

The above and other embodiments and features of this invention will be still further apparent from the ensuing description and appended claims.

### BRIEF DESCRIPTION OF THE DRAWING

FIG. 1 is a schematic line drawing of a loop reactor system used in the practice of this invention.

### FURTHER DETAILED DESCRIPTION

As used herein including the claims:

1) The term “reaction-derived” means that the composition of the product is reaction determined and not the result of use of downstream purification techniques, such as recrystallization or chromatography, or like procedures that can affect the chemical composition of the product. Adding water or an aqueous base such as sodium hydroxide to the reaction mixture to inactivate the catalyst, and washing away of non-chemically bound impurities by use of aqueous washes such as with water or dilute aqueous bases are not excluded by the term “reaction-derived”. In other words, the products are directly produced in the synthesis process without use of any subsequent procedure to remove or that removes nonabromodiphenylalkane from decabromodiphenylalkane.

2) The term “high purity” especially as applied to decabromodiphenylethane means that the reaction-derived DBDPE product comprises more than 97% of DBDPE with the balance consisting essentially of octabromodiphenyl ethane (Br₈DPE) and/or nonabromodiphenyl ethane (Br₉DPE) with the amount of Br₈DPE being less than the amount of Br₉DPE. Preferred reaction-derived DBDPE product comprises at least 98% of DBDPE and more preferred reaction-derived DBDPE product comprises at least 99% DBDPE, in both cases, with the balance consisting essentially of Br₈DPE and Br₉DPE, again with the amount of Br₉DPE exceeding the amount of Br₈DPE. In even higher purity product, nonabromodiphenylethane may be the only impurity present with no detectable amount of octabromodiphenylethane being present.

For the purposes of this invention, unless otherwise indicated, the % values given for DBDPE and nonabromodiphenyl ethane are to be understood as being the area % values that are derived from gas chromatography analysis. A procedure for conducting such analyses is presented hereinafter.

In the processes of this invention diphenylalkane is brominated in a loop reactor containing at least a liquid-phase comprised of bromine, and preferably containing an excess of bromine which is maintained in the circulating inventory in the reactor. Also Lewis acid bromination catalyst is typically in the circulating inventory in the reactor. An important feature of the invention is that the diphenylalkane and/or partially-brominated diphenylalkane is fed, continuously or periodically into the loop reactor at a suitable entry locus, preferably by means of an injector nozzle, so that bromination of the feed is promptly initiated and conducted as the liquid phase of the reaction mixture passes along the path defined by the loop reactor.

During the bromination reaction taking place in the loop reactor bromine is consumed and Lewis acid catalyst is consumed and/or depleted and thus it is preferred to replenish the bromine and catalyst either periodically or preferably, continuously. One or two inlets on the loop reactor are provided for this purpose. It is usually more convenient to feed both of these components as a mixture in suitable proportions using a single inlet.

In one embodiment of the invention, the process is conducted such that complete bromination to decabromodiphenylalkane occurs within less than a full cycle of travel of the circulating inventory through the loop of the reactor. In this embodiment elevated bromination temperatures and slow rates of travel are utilized in a loop reactor having a long cycle of travel so that the bromination reaction goes to completion in the circulating inventory within one cycle through the loop, starting from the locus of diphenylalkane feed and ending before again reaching that locus. During this less than one loop of travel, the decabromodiphenylalkane forms as particulate solids and is carried along as part of the inventory traveling in the reactor. Thus in this embodiment the solids are removed from the circulating inventory before the locus of diphenylalkane feed is reached. For this purpose a filter and solids collector is disposed to receive the circulating inventory at a suitable location upstream from the locus of diphenylalkane feed so that the solids are removed from the circulating inventory and are collected. Meanwhile the liquid phase continues its travel and when it reaches the locus of diphenylalkane feed, fresh diphenylalkane is added and a second cycle ensues. If the feed of diphenylalkane is continuous and uniform rates of (i) feed, (ii) travel and (iii) filtration are maintained, all of the foregoing activities will continuously take place at more or less the same locations within the loop reactor.

A modification of the above embodiment involves using a discontinuous rate of diphenylalkane feed whereby one charge of diphenylalkane is made and the circulating inventory carries that charge throughout the loop more than once while either bypassing the filter and solids collector or while the filter and solids collector is deliberately inactivated, so that the portion of the circulating inventory carrying the feed undergoes bromination during one or more cycles of travel throughout the loop before the circulating inventory is either directed back through the filter and solids collector or the filter and solids collector is reactivated. This embodiment preferably utilizes automated switching mechanisms to properly time, initiate and discontinue the periodic pulses of feed and to properly time, initiate and discontinue the periodic bypassing or periodic inactivation and reactivation of the filter and solids collector.

In another embodiment of the invention (i) the feed of diphenylalkane to the circulating inventory in the loop reactor is continuous, (ii) the circulation rate of the circulating inventory is constant and substantially uniform throughout the loop, (iii) a substantially constant bromination temperature is maintained, and (iv) the replenishment of bromine and catalyst is conducted to maintain a substantially constant amount of these components in the circulating inventory. By suitably coordinating these conditions, the bromination of the diphenylalkane operates under steady state conditions. This in turn results in an operation in which the solids which form in the liquid phase are reaction-derived decabromodiphenylalkane product of high purity and the solids can be allowed to circulate within the loop while continuously filtering off a portion of the solids from the circulating inventory.

As noted above, the process technology of this invention is deemed applicable to the bromination of diphenylalkanes, i.e., compounds which can be depicted by the formula:

Ph-R-Ph

where Ph is a phenyl group and R is a straight chain alkaline group containing in the range of 1 to about 12 carbon atoms, preferably 1 to 6 carbon atoms, and more preferably the alkaline group has 2 carbon atoms (i.e., this more preferred reactant is 1,2-diphenylethane which is more commonly known as diphenylethane). Non-limiting examples of 1,2-diphenylalkanes which may be used as reactants in the processes of this invention include diphenylmethane, 1,3-diphenylpropane, 1,4-diphenylbutane, 1,3-diphenyl(2-methylpropane), 1,5-diphenylpentane, 1,6-diphenylhexane, 1,5-diphenyl(3-methylpentane), 1,4-diphenyl(2-methylpentane) and analogous compounds.

This invention is also deemed applicable to the bromination of partially brominated diphenylalkanes which are compounds of the above formula in which, as individual compounds, one of the Ph groups is, or both of the Ph groups are, partially brominated. In the case of mixtures, a wider range of partial bromination can exist. Thus on the low side, typically some of the Ph groups in the mixture of diphenylalkanes have one bromine atom on one Ph group. On the higher side, greater amounts of bromine substitution can exist on all Ph groups in the mixture. In any case the extent of partial bromination will usually be up to a total of about 4 bromine atoms per molecule.

For convenience, the ensuing description will refer more specifically to bromination of diphenylethane. It is to be understood, however, that the principles apply to bromination of diphenylalkanes, and that the reaction conditions can be generally applied to the bromination of other diphenylalkanes.

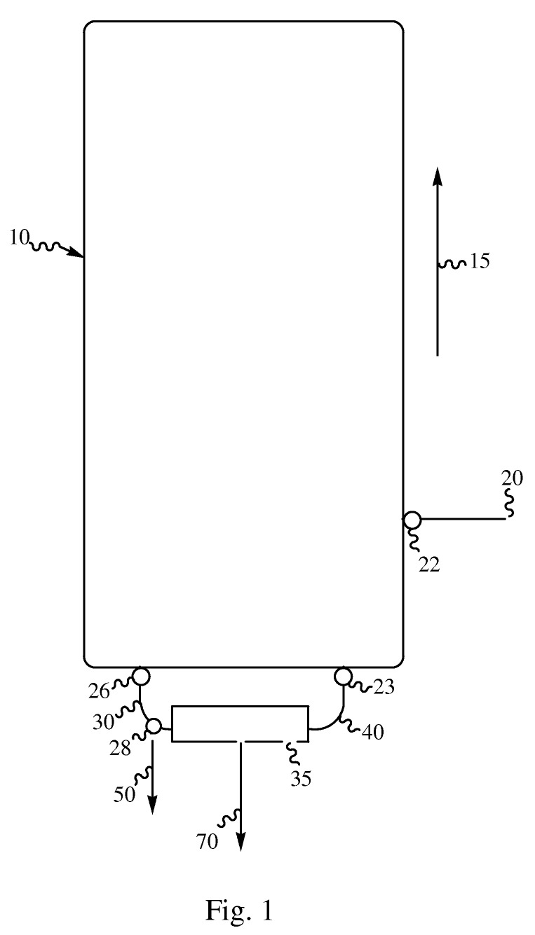

FIG. 1 illustrates in schematic fashion one type of loop reactor system that can be used in practicing this invention. The loop designated generally as 10 is typically aligned in a plane which can be vertical, inclined at an angle, or horizontal. Horizontal or substantially horizontal alignment of the loop is generally preferred. At start up, loop 10 is charged with liquid solvent such as dibromomethane and optionally, bromine, via feed line 20 with gate valve 22 open. The amount of these components charged is typically an amount which occupies in the range of about 15 to about 30 percent of the total volume of loop 10. With gate valves 22 and 23 and take-off valve 26 all closed, the pump(s) (not shown) is/are put in operation to cause circulation of this liquid phase mixture within the loop in the direction of arrow 15. Then gate valve 22 in feed line 20 is opened and simultaneously a mixture of diphenylethane and/or partially brominated diphenylethane, liquid bromine, and a catalytic quantity of Lewis acid catalyst such as aluminum chloride is injected continuously into the liquid mixture flowing in the loop. Bromination promptly occurs in the inventory flowing in the loop downstream from feed line 20. Depending on the length of the loop 10 and the rate at which the inventory is flowing therein, decabromodiphenylethane solids can form before any portion of the inventory containing such solids reaches take-off line 30. With take-off valve 26 open, gate valve 23 closed, and two-way valve 28 open only to discharge line 50, a portion of the inventory flows into line 30 and at start-up, out through discharge line 50 with the remainder of the inventory continuing to flow through loop 10. In this way, the initial quantity of dibromomethane solvent and initial portions of the reaction product mixture can be discarded until the system reaches a steady-state condition. Once a steady-state condition is reached, gate valve 23 is opened, and two-way valve 28 is closed to discharge line 50 and opened to filter 35 so that a portion of the inventory flows through filter 35, which removes the decabromodiphenylethane solids from the inventory and discharges that product as illustrated by line 70. The filtrate from filter 35 flows back into loop 10 via return line 40 and through open gate valve 23 in return line 40. After start-up the amount of diphenylethane and/or partially brominated diphenylethane, liquid bromine and Lewis acid bromination catalyst entering loop 10 can thereafter be controlled or regulated by gate valve 22 so as to maintain a constant or substantially constant volume of inventory flowing in the overall system as well as a proper amount of these incoming components in relation to the amounts of bromine and catalyst being consumed and the amount of product solids being withdrawn from the system as indicated by line 70.

Hydrogen bromide coproduct can be removed from the system at any suitable location and processed in any of a variety of known ways. One preferred way of handling the HBr is to provide a take-off line (not shown) in the loop 10 downstream from gate valve 22 which receives and transmits a gaseous mixture of bromine and hydrogen bromide. This mixture is passed into a condenser (not shown) which cools the mixed gases and condenses the bromine into liquid form which is returned to loop 10. The gaseous HBr is then passed into a scrubber (not shown) which contains either water whereby hydrobromic acid is produced, or a base such as sodium hydroxide or calcium hydroxide whereby sodium bromide or calcium bromide is formed. All such products produced from the HBr gas are useful as articles of commerce.

The system depicted in, and described with reference to, FIG. 1 is merely an illustration of one way of conducting a process of this invention. It will be readily apparent to those of ordinary skill in the art that the system depicted in FIG. 1 can be modified in various ways in accordance with this invention as described elsewhere in this document. As just one example, take off valve 26 can be replaced in about the same location by a valve (not shown) in loop 10 itself which valve either (i) allows a portion of the inventory to continue to flow in loop 10 and a portion to flow into take-off line 30 and, with two-way valve 28 open only to filter 35, thence into filter with the filtrate passing from the filter through return line 40 and through open gate valve 23 into loop 10, or (ii) opens only to take-off line 30 so that with two-way valve 28 open only to filter 35, all of the traveling inventory flows into filter with the filtrate passing from the filter through return line 40 and through open gate valve 23 and back into loop 10.

The temperature at which the bromination occurs can be varied but preferably is in at an elevated temperature at which the bromine remains in the liquid state under the autogenous pressure in the loop reactor. Typically temperatures in the range of about 55 to about 80° C. are used, but departures from this range are permissible and within the contemplation and scope of this invention. If desired, the loop can be segmented so that the pressure in the regions where active bromination occurs can be regulated and if necessary, the temperature of the exothermic reaction can be controlled by indirect heat exchange.

The coproduct in the reaction, hydrogen bromide, is typically released in part in the form of a vapor. For reasons of economy of operation it is desirable to recover the coproduct hydrogen bromide such as by passing the vapors into a scrubbing system in which the hydrogen bromide is converted either to hydrobromic acid using water as the scrubbing liquid, or into a hydrobromic acid salt using an aqueous solution of metal base such as aqueous sodium hydroxide as the scrubbing liquid.

This invention is deemed to enable the preparation of highly pure DBDPE products that are derived from the bromination of diphenyl ethane. Such products can be said to be “reaction-derived” since they are reaction determined and not the result of use of downstream purification techniques, such as recrystallization, chromatography, or like procedures. In other words, the products of high purity are directly produced in the synthesis process apart from use of subsequent purification procedures that remove nonabromodiphenyl ethane from the decabromodiphenylethane product.

In the embodiments of this invention, 1,2-diphenylethane (also called dibenzyl or bibenzyl) is used. The term “diphenylethane” as used throughout this document means 1,2-diphenylethane unless otherwise noted. The DPE can be fed separately to the loop in molten form or as a solution in an appropriate solvent such as dibromomethane or in bromine itself, but preferably the feed is in the form of a solution in bromine which also contains suspended or dissolved Lewis acid catalyst.

Excess bromine is used in the Lewis acid catalyzed bromination reaction. Typically, the reaction mixture traveling in the loop reactor will contain in the range of at least about 14 moles of bromine per mole of DPE fed and/or being fed thereto, and preferably, the reaction mixture contains in the range of about 16 to about 25 moles of bromine per mole of DPE fed and/or being fed thereto. It is possible to use more than 25 moles bromine per mole of DPE in order to provide an even greater reserve of bromine to also serve as solvent for the reaction.

Various iron and/or aluminum Lewis acids can be added to the bromine and/or to the reaction mixture to serve as the bromination catalyst. These include the metals themselves such as iron powder, aluminum foil, or aluminum powder, or mixtures thereof. Preferably use is made of such catalyst materials as, for example, ferric chloride, ferric bromide, aluminum chloride, aluminum bromide, or mixtures of two or more such materials. More preferred are aluminum chloride and aluminum bromide with addition of aluminum chloride being more preferred from an economic standpoint. It is possible that the makeup of the catalyst may change when contained in a liquid phase of refluxing bromine. For example, one or more of the chlorine atoms of the aluminum chloride may possibly be replaced by bromine atoms. Other chemical changes are also possible. The Lewis acid should be employed in an amount sufficient to effect a catalytic effect upon the bromination reaction being conducted. Typically, the amount of Lewis acid used will be in the range of about 0.06 to about 2 wt %, and preferably in the range of about 0.2 to about 0.7 wt % based on the weight of the bromine being used.

A residence period in the loop reactor in the range of about 15 to about 90 minutes and preferably in the range of about 30 to about 60 minutes is recommended. However, departures from these ranges are permissible and are within the contemplation and scope of this invention.

As noted above, the product formed in the bromination reaction is typically recovered from the circulating inventory in the loop reactor by use of filtration. However, the system can be configured to recover the products solids by other physical separation procedures such as by centrifugation or decantation.

The separated product is typically washed with water or dilute aqueous bases in order to wash away non-chemically bound impurities. It is then subjected to finishing operations such as heating to remove free bromine and grinding to convert the product to a uniform particle size before packaging.

In order to determine the composition of the brominated product formed in a process of this invention, a gas chromatographic procedure is used. The gas chromatography is conducted on a Hewlett-Packard 5890 Series II gas chromatograph (or equivalent) equipped with a flame ionization detector, a cool on-column temperature and pressure programmable inlet, and temperature programming capability. The column is a 12QC5 HTS capillary column, 12 meter, 0.15μ film thickness, 0.53 mm diameter, part number 054657, available from SGE, Inc. (2007 Kramer Lane, Austin, Tex. 78758). Conditions are: detector temperature 350° C.; inlet temperature 70° C.; heating at 125° C./min to 350° C. and holding at 350° C. until the end of the run; helium carrier gas at 10 ml/min.; inlet pressure 4.0 psi, increasing at 0.25 psi/min. to 9.0 psig and holding at 9.0 psi until the end of the run; oven temperature 60° C. with heating at 12° C./min. to 350° C. and holding for 10 min.; and injection mode of cool on-column. Samples are prepared by dissolving, with warming, 0.003 grams in 10 grams of dibromomethane and injection of 2 microliters of this solution. The integration of the peaks is carried out using Target Chromatography Analysis Software from Thru-Put Systems, Inc. (5750 Major Blvd., Suite 200, Orlando, Fla. 32819; currently owned by Thermo Lab Systems). However, other and commercially available software suitable for use in integrating the peaks of a chromatograph may be used.

The decabromodiphenylalkane products formed in processes of this invention are white or slightly off-white in color. White color is advantageous as it simplifies the end-users task of insuring consistency of color in the articles that are flame retarded with such products.

The decabromodiphenylalkane products formed in the processes of this invention may be used as flame retardants in formulations with virtually any flammable material. The material may be macromolecular, for example, a cellulosic material or a polymer. Illustrative polymers are: olefin polymers, cross-linked and otherwise, for example homopolymers of ethylene, propylene, and butylene; copolymers of two or more of such alkene monomers and copolymers of one or more of such alkene monomers and other copolymerizable monomers, for example, ethylene/propylene copolymers, ethylene/ethyl acrylate copolymers and ethylene/propylene copolymers, ethylene/acrylate copolymers and ethylene/vinyl acetate copolymers; polymers of olefinically unsaturated monomers, for example, polystyrene, e.g. high impact polystyrene, and styrene copolymers, polyurethanes; polyamides; polyimides; polycarbonates; polyethers; acrylic resins; polyesters, especially poly(ethyleneterephthalate) and poly(butyleneterephthalate); polyvinyl chloride; thermosets, for example, epoxy resins; elastomers, for example, butadiene/styrene copolymers and butadiene/acrylonitrile copolymers; terpolymers of acrylonitrile, butadiene and styrene; natural rubber; butyl rubber and polysiloxanes. The polymer may be, where appropriate, cross-linked by chemical means or by irradiation. The decabromodiphenylalkane products formed in a process of this invention can also be used in textile applications, such as in latex-based back coatings.

The amount of a decabromodiphenylalkane product formed pursuant to this invention used in a formulation will be that quantity needed to obtain the flame retardancy sought. In general, the formulation and resultant product may contain from about 1 to about 30 wt %, preferably from about 5 to about 25 wt % of decabromodiphenylalkane product of this invention. Master batches of polymer containing decabromodiphenylalkane, which are blended with additional amounts of substrate polymer, typically contain even higher concentrations of decabromodiphenylalkane, e.g., up to 50 wt % or more.

It is advantageous to use the DBDPE products formed pursuant to this invention in combination with antimony-based synergists, e.g. Sb₂O₃. Such use is conventionally practiced in all DBDPE applications. Generally, the DBDPE products of this invention will be used with the antimony based synergists in a weight ratio ranging from about 1:1 to 7:1, and preferably of from about 2:1 to about 4:1.

Any of several conventional additives used in thermoplastic formulations may be used, in their respective conventional amounts, with the DBDPE products of this invention, e.g., plasticizers, antioxidants, fillers, pigments, UV stabilizers, etc.

Thermoplastic articles formed from formulations containing a thermoplastic polymer and DBDPE product of this invention can be produced conventionally, e.g., by injection molding, extrusion molding, compression molding, and the like. Blow molding may also be appropriate in certain cases.

Components referred to by chemical name or formula anywhere in the specification or claims hereof, whether referred to in the singular or plural, are identified as they exist prior to coming into contact with another substance referred to by chemical name or chemical type (e.g., another component, a solvent, or etc.). It matters not what chemical changes, transformations and/or reactions, if any, take place in the resulting mixture or solution as such changes, transformations, and/or reactions are the natural result of bringing the specified components together under the conditions called for pursuant to this disclosure. Thus the components are identified as ingredients to be brought together in connection with performing a desired operation or in forming a desired composition. Also, even though the claims hereinafter may refer to substances, components and/or ingredients in the present tense (“comprises”, “is”, etc.), the reference is to the substance, component or ingredient as it existed at the time just before it was first contacted, blended or mixed with one or more other substances, components and/or ingredients in accordance with the present disclosure. The fact that a substance, component or ingredient may have lost its original identity through a chemical reaction or transformation during the course of contacting, blending or mixing operations, if conducted in accordance with this disclosure and with ordinary skill of a chemist, is thus of no practical concern.

Except as may be expressly otherwise indicated, the article “a” or “an” if and as used herein is not intended to limit, and should not be construed as limiting, a claim to a single element to which the article refers. Rather, the article “a” or “an” if and as used herein is intended to cover one or more such elements, unless the text expressly indicates otherwise.

Each and every patent or publication referred to in any portion of this specification is incorporated in toto into this disclosure by reference, as if fully set forth herein.

This invention is susceptible to considerable variation in its practice. Therefore the foregoing description is not intended to limit, and should not be construed as limiting, the invention to the particular exemplifications presented hereinabove.

That which is claimed is:

1. A process for preparing reaction-derived decabromodiphenylalkane product of high purity, which process comprises:
A) maintaining in a loop reactor a circulating inventory comprising at least liquid bromine and Lewis acid bromination catalyst;
B) introducing diphenylalkane and/or partially brominated diphenylalkane into said loop reactor at a reactor inlet portion so that bromination occurs in said loop reactor; and
C) after a period of travel in said loop reactor that enables formation therein of reaction-derived decabromodiphenylalkane product of high purity in the form of solids, removing via an outlet portion in the loop reactor a portion of the circulating inventory containing at least some of said solids, and having the remaining portion of the circulating inventory continue flowing in the loop reactor.

2. A process as in claim 1 further comprising maintaining in the circulating inventory a stoichiometric excess of liquid bromine relative to diphenylalkane and/or partially brominated diphenylalkane (ii) a catalytic quantity of Lewis acid bromination catalyst, by continuously or periodically introducing into the loop reactor (i) fresh liquid bromine to replenish the bromine consumed in the bromination and/or removed from the reactor and (ii) fresh Lewis acid bromination catalyst to replenish the catalyst consumed in the bromination and/or removed from the reactor.

3. A process as in claim 1 further comprising separating solids from the portion of the circulating inventory that has passed into said outlet portion.

4. A process as in claim 3 wherein said solids are separated by filtration and wherein said process further comprises having the liquid filtrate from said filtration return to the loop reactor.

5. A process as in any of claims 1-4 wherein the Lewis acid bromination catalyst as charged to the circulating inventory is in the form of subdivided iron, subdivided aluminum, aluminum foil, ferric chloride, ferric bromide, aluminum chloride, aluminum bromide, or a combination of any two or more of the foregoing.

6. A process as in any of claims 1-4 the Lewis acid bromination catalyst is introduced into the circulating inventory in the form of an aluminum trihalide in which the halogen atoms are chlorine and/or bromine.

7. A process for preparing reaction-derived decabromodiphenylethane of high purity, which process comprises:
A) introducing diphenylethane or partially brominated diphenylethane, or both, into a loop reactor containing a circulating inventory comprising at least (a) liquid bromine, (b) Lewis acid bromination catalyst and optionally (c) bromination reaction products formed by bromination of said partially brominated diphenylethane;
B) separating a portion of the circulating inventory from said reactor at a remote locus downstream from the locus of introduction of the diphenylethane or partially brominated diphenylethane, or both, and recovering reaction-derived product solids from the separated portion of the circulating inventory and returning to the loop reactor, liquid from which said solids have been removed, and
C) replenishing bromine and Lewis acid bromination catalyst in the circulating inventory so as to maintain therein (i) an excess of bromine relative to the incoming diphenylethane and (ii) a catalytic quantity of Lewis acid bromination catalyst.

8. A process as in claim 7 wherein said recovered reaction-derived product solids are contacted and/or washed with water, an aqueous base, or both.

9. A process for preparing reaction-derived decabromodiphenylethane of high purity, which process comprises:
A) introducing diphenylethane or partially brominated diphenylethane, or both, into a loop reactor containing a circulating inventory comprising at least liquid bromine and Lewis acid bromination catalyst;
B) recovering decabromodiphenylethane product solids from the circulating inventory at a remote locus downstream from the locus of introduction of diphenylethane or partially brominated diphenylethane, or both, and enabling liquid from which said solids have been removed to continue flowing in said reactor as part of the circulating inventory contained within said reactor; and
C) replenishing bromine and Lewis acid bromination catalyst in the circulating inventory so as to maintain therein (i) an excess of bromine relative to the incoming diphenylethane and (ii) a catalytic quantity of Lewis acid bromination catalyst.

10. A process as in any of claims 7-9 wherein the Lewis acid bromination catalyst is introduced into the circulating inventory in the form of an aluminum trihalide in which the halogen atoms are chlorine and/or bromine.
